(19) United States
(12) Patent Application Publication
(10) Pub. No.: US 2004/0202705 A1
(43) Pub. Date: Oct. 14, 2004

(54) Transdermal administration of huperzine

(75) Inventors: Weihong Xiong, Salt Lake City, UT (US); Dinesh C. Patel, Salt Lake City, UT (US)

Correspondence Address: M. Wayne Western, THORPE NORTH & WESTERN, LLP, P.O. Box 1219, Sandy, UT, 84091-1219, US

(73) Assignee: Xel Herbaceucticals, Inc.

(21) Appl. No.: 10/723,435

(22) Filed: Nov. 26, 2003

Related U.S. Application Data
(63) Continuation of application No. 09/705,286, filed on Nov. 2, 2000, now abandoned.
(60) Provisional application No. 60/163,636, filed on Nov. 4, 1999.

(51) Int. Cl. A61K 9/70

(52) U.S. Cl. 424/449; 514/291

(57) ABSTRACT

The present invention provides a composition of transdermally administered huperzine for improving memory and cognitive function. In one aspect, huperzine is delivered in a sufficient amount to achieve and maintain a blood plasma Huperzine level of about 0.5 ng/mL to about 30 ng/mL. Huperzine may be delivered by itself, or in combination with other elements, such as additional cholinesterase inhibitors, drugs or treatment agents, or positive health promoting substances. Various formulations for the transdermal delivery of huperzine are disclosed, and may include selected penetration enhancers.

### PRIORITY DATA

This application is a continuation of U.S. patent application Ser. No. 09/705,286, filed on Nov. 2, 2000, which claims priority to provisional U.S. patent application serial No. 60/163,636 which was filed on Nov. 4, 1999, each of which is incorporated herein by reference.

### FIELD OF THE INVENTION

The present invention relates generally to compositions and methods for improving memory and cognitive function in humans. More particularly, it concerns a composition and method for transdermally administering huperzine, and achieving a desired huperzine blood plasma level.

### BACKGROUND OF THE INVENTION

Good memory skills and cognitive function are tantamount to an individual's independence and ability to be self-sustaining. Further, good memory skills and cognitive function are fundamental factors contributing to the quality of a person's life. Often, those individuals with superior cognitive function and memory are more productive and able to excel in academic and occupation arenas. Additionally, individuals who are able to think clearly and exercise sound judgment may find a higher quality of relationships with others.

While many individuals enjoy an acceptable level of cognitive function and memory ability, many desire to improve these aspects of their physiology. Additionally, many individuals are afflicted with a level of cognitive function and memory performance, which hinders them from effectively interacting with others, or from viably competing and excelling in certain aspects of life. In many instances, individuals may be so severely afflicted with poor cognitive function and memory ability as to be considered mentally disabled.

One form of mental disability is the affliction known as Alzheimer's Disease (AD). AD has been reported to be the current leading cause of loss of independent living and subsequent institutionalization. Other mental disabilities including myasthenia gravis, which results from aging and Down's syndrome may dictate the loss of an individual's independence.

Acetylcholinersterase (AChE) or cholinesterase inhibitors have now been found to abate memory loss and cognitive degeneration in many cases. Presently, two actylcholinesterase (AChE) inhibitor drugs, Tacrine and Donepezil, have been approved for the treatment of AD. Tacrine has a moderate beneficial effect on the deterioration of cognition, but also results in side effects such as hepatotoxicity. Reversible hepatotoxicity has been observed in approximately 30% of patients afflicted with AD after treatment with therapeutic doses of Tacrine. Such a side effect limits the clinical value of this substance. Therefore, and AChE inhibitor which provides a significant memory and cognition improving effect, with minimal toxicity is highly desirable.

One difficulty in treating individuals experiencing memory loss and cognitive impairment with many forms of medication, such as oral dosage forms, is the frequency of required administration. Patient compliance with a highly frequent dosage regimen has traditionally been less than satisfactory even with those people having adequate memory abilities, let alone those with varying forms of cognitive dysfunction. Therefore, a sustained release formulation for delivering a cognitive function and memory improving substance, which requires only periodic administration is very desirable.

### SUMMARY OF THE INVENTION

Huperzine is a natural, potent, and selective cholinesterase inhibitor. As a Lycopodium alkaloid, huperzine is found in the club moss Huperzia Serrata, also known as Quian Ceng Ta, and has been used for centuries in Chinese herbal medicine to treat a variety of ailments and disorders such as fever and inflammation. Huperzine has also been prescribed in China for the amelioration of memory loss, dementia, and cognitive function disorders. The use of aiding memory and cognitive function for huperzine is documented in The Merck Index, 12^{th} Ed. (1999).

Research has shown that huperzine helps to alleviate memory loss problems and cognitive function disorders due a variety of causes, including but not limited to aging, AD, and other afflictions such as Auto Immune Deficiency Syndrome (AIDS). Further, research has shown that healthy individuals may enhance their memory and cognitive function by the administration of huperzine, and that huperzine may be effective for preventing the deterioration of cognitive function and memory ability. Other conditions which huperzine administration may ameliorate or improve include Apathy-Motivation Syndromes, such as Schizophrenia and Parkinson's disease, Behavioral Syndromes, such as agitation, aggression, and depression, Down's Syndrome, Dementia, Fatigue Syndrome, Frontal Lobe Syndrome, Glaucoma, Multiple Sclerosis, Myasthenia Gravis, and Reward Deficiency Syndrome.

The inhibition of cholinesterase produced by huperzine may be performed long-term, without significant side effects, and is reversible. Additionally, it has been shown that huperzine also increases the synthesis and release of acetylcholine in the brain. This dual action of inhibiting cholinesterase and facilitating of the synthesis and release of acetylcholine may be accomplished with therapeutic doses of huperzine. Additionally, the level of huperzine required to effect therapeutic results produces only mild side effects. In addition to the inhibition of cholinesterase and the facilitation of the synthesis and release of acetylcholine, huperzine has been shown to have neuro-protective abilities. Specifically, when administered at therapeutic levels, huperzine arrests the damage incurred by nerve cells due to the effects of glutamate toxicity. Glutamate is an excitatory (stimulatory) neuro-transmitter. During a stroke or other brain injury, excess glutamate is released in the brain, triggering the additional release of certain enzymes inside nerve cells that lead to cell damage and death. Therefore, because of its nerve cell protective ability, huperzine may be useful in ameliorating the effects due to strokes, epilepsy, and other neurological disorders.

Accordingly, in the present invention provides a transdermal formulation for improving memory and cognitive function. In one aspect, the transdermal formulation includes an amount of huperzine, which is sufficient to achieve a huperzine blood plasma level of from about 0.1 to about 30 ng/ml, an inert carrier and, a permeation enhancer selected from the group consisting of: fatty acids, fatty acid esters, fatty alcohols, fatty acid esters of lactic acid, fatty acid esters of glycolic acid, amides, amines, pyrrolidones, glycerol trimesters, terpenes, surfactants, complexing agents, biologics, their salts, and mixtures thereof. In another aspect, the blood plasma concentration of huperzine achieved is from about 1 to about 15 ng/ml. In another aspect, the transdermal formulation achieves the blood plasma level of from about 0.1 to about 30 ng/ml within about 0.5 to about 10 hours after administration of the formulation.

The transdermal formulation may be configured to provide an extended or sustained huperzine release. In one aspect, a single dosage of the transdermal formulation may be sufficient to achieve and sustain the huperzine blood plasma level of from about 0.1 to 30 ng/ml for a duration of at least about 3 days. In another aspect, a single dosage of the transdermal formulation may be sufficient to sustain the huperzine blood plasma level of from about 0.1 to about 30 ng/ml for at least about 7 days.

Various types of huperzine may be effective in improving cognitive function and memory. In one aspect, the huperzine may be a member selected from the group consisting of huperzine A, huperzine B, huperzine X, their salts, analogs, derivatives, prodrugs, and mixtures thereof. In another aspect, the huperzine may be huperzine A. In another aspect, the huperzine may be huperzine B. In yet another aspect, the huperzine may be huperzine X.

In addition to huperzine, the transdermal formulation of the present invention may include additional cholinesterase inhibitors, which are co-delivered with the huperzine. In another aspect, additional cholinesterase inhibitors may be synthesized with huperzine to produce a huperzine hybrid agent. In one aspect, the hybrid agent may be a huperzine-tacrine hybrid.

The transdermal formulation of the present invention may also contain various other positive health-imparting agents. In one aspect, the health imparting agent may be a member selected from the group consisting of: vitamins, minerals, amino acids, herbal and botanical extracts, anti-oxidants, and mixtures thereof. In another aspect, the health-imparting agent may be a vitamin. In a further aspect, the health-imparting substance may be a mineral. In yet another aspect, the health-imparting agent may be an amino acid. In yet another aspect, the health-imparting agent may be an herbal extract. In another aspect of the invention, the health-imparting agent may be a botanical extract. In a further aspect of the invention, the health-imparting substance may be an anti-oxidant.

The transdermal formulation of the present invention may include other drugs, or treatment agents, which treat disorders often closely associated with memory loss. By way of example without limitation, in one aspect, the formulation may include one or more antipsychotics agents. In another aspect, the formulation may include one or more anxiolytic agents. In yet another aspect, the formulation may include one or more antidepressants agents. In a further aspect, the formulation may include one or more hormones.

Various transdermal formulations may be used as part of the present invention for transdermally delivering huperzine. In one aspect, the transdermal formulation may be a topical formulation. In another aspect, the transdermal formulation may be an adhesive matrix patch. In yet another aspect, the transdermal formulation may be a liquid reservoir system, or patch.

While the transdermal formulation of the present invention may include a variety of enhancers, no enhancer is necessary in order to achieve the desired blood plasma levels in many instances. Therefore, in one aspect the transdermal formulation of the present invention may be free of an enhancer, and consist essentially of an amount of huperzine sufficient to achieve a huperzine blood plasma level of from about 0.1 to about 30 ng/ml admixed with an inert carrier. In a further aspect, the above-recited good health-imparting substances recited above may be added to the mixture of huperzine and carrier.

In addition to a huperzine-containing transdermal formulation, the present invention encompasses a method of improving memory and cognitive function. In one aspect, the method includes transdermally administering an amount of huperzine sufficient to achieve a huperzine blood plasma level of from about 0.1 to about 30 ng/ml. In another aspect, the transdermal administration of huperzine is sufficient to achieve a huperzine blood plasma level of from about 1 to about 15 ng/ml. In yet another aspect, the huperzine blood plasma level is achieved within about 0.5 to about 10 hours after initiation of the huperzine administration. In a further aspect, the huperzine blood plasma level of about 0.1 to about 30 ng/ml is sustained for a period of at least 3 days from a single transdermal administration. In another aspect, the huperzine blood plasma level is sustained for a period of at least 7 days from a single transdermal administration.

The method of the present invention encompasses the co-delivery of huperzine and addition cholinesterase inhibitors. In one aspect, the additional cholinesterase inhibitor may be synthesized with huperzine to create a huperzine hybrid compound. In another aspect, the huperzine hybrid compound may be huperzine-tacrine.

Further, the method of the present invention includes delivery of drugs, or treatment agents other than huperzine that may be administered concomitantly with the huperzine. In one aspect, a treatment agent may be a bioactive substance, which is effective against a disease, or condition that is closely related to, or the cause of memory loss, or cognitive function impairment. Examples of treatment agents include, but are not limited to antipsychotics, anxiolytics, antidepressants, hormones, and mixtures thereof.

Further, the method of the present invention encompasses the co-delivery of huperzine and an additional good health-imparting agent. In one aspect, the transdermal formulation may be a topical formulation. In another aspect, the transdermal formulation may be an adhesive matrix patch. In yet another aspect, the transdermal formulation may be a liquid reservoir system, or patch.

The method of the present invention also encompasses the co-delivery of huperzine and other good-health imparting agents. In one aspect, the health imparting agent may be a member selected from the group consisting of: vitamins, minerals, amino acids, herbal and botanical extracts, anti-oxidants, and mixtures thereof. In another aspect, the health-imparting agent may be a vitamin. In a further aspect, the health-imparting substance may be a mineral. In yet another aspect, the health-imparting agent may be an amino acid. In yet another aspect, the health-imparting agent may be an herbal extract. In another aspect of the invention, the health-imparting agent may be a botanical extract. In a further aspect of the invention, the health-imparting substance may be an anti-oxidant.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### DETAILED DESCRIPTION

Before the present formulation and method for achieving specified huperzine blood plasma levels are disclosed and described, it is to be understood that this invention is not limited to the particular process steps and materials disclosed herein, but is extended to equivalents thereof as would be recognized by those ordinarily skilled in the relevant arts. It should also be understood that terminology employed herein is used for the purpose of describing particular embodiments only and is not intended to be limiting.

A. Definitions

In describing and claiming the present invention, the following terminology will be used.

The singular forms “a,” and, “the” include plural referents unless the context clearly dictates otherwise. Thus, for example, reference to a huperzine delivery device containing “a delivery substance” includes a mixture of two or more delivery substances, reference to “an adhesive” includes reference to one or more of such adhesives, and reference to “an excipient” includes reference to a mixture of two or more of such excipients.

As used herein, the term “huperzine,” or “huperzines” may be used interchangeably and refer to huperzine A, B, and X, their analogues, derivatives, salts and prodrugs, and mixtures thereof, whether synthesized or extracted as a natural product from a natural huperzine source, or whether partially extracted from a natural source and further synthesized.

As used herein, “cholinesterase,” and “acetyl cholinesterase,” may be used interchangeably, and refer to any enzyme that catalyzes the hydrolysis of choline esters. For example, acetyl cholinesterase facilitates the hydrolysis of acetylcholine by into acetic acid and choline.

As used herein, “positive health benefit conveying, or imparting agent” and similar expressions refer to any substance either synthesized or extracted from a natural source, which is beneficial to the human body when imparted thereto. Examples of general positive health benefit conveying substances include, but are not limited to vitamins, minerals, anti-oxidants, amino acids, botanical and herbal extracts, and good memory promoting substances other than huperzine.

As used herein, “treatment agent” or “drug” may be used interchangeably, and refer to a physiologically active substance other than huperzine, or other cholinesterase inhibitors, which may be used to treat or improve a physiological condition. Examples of treatment agents include, but are not limited to: antipsychotics, anxiolytics, antidepressants, hormones, and mixtures thereof.

As used herein, “huperzine delivery formulation,” “transdermal delivery formulation,” or “transdermal formulation” refers to any huperzine containing device, system, product, chemical combination, or mechanism capable of being applied to, or against the skin, to effect transdermal delivery, of huperzine.

As used herein, the term “skin” refers to any membrane of the human body to which a chemical formulation or composition may be applied including the external skin of the body, the mucosa membranes of the nasal, oral, vaginal, and rectal cavities.

As used herein, the term “transdermal” or “percutaneous” delivery means delivery of a substance or agent, by passage into and through the skin. Hence the terms “transdermal” and “transmucosal” are used interchangeably unless specifically stated otherwise. Likewise, the terms “skin”, “derma”, “epidermis”, “mucosa”, and the like shall also be used interchangeably unless specifically stated otherwise.

As used herein, the terms “enhancement”, “penetration enhancement”, or “permeation enhancement” refer to an increase in the permeability of the skin, to a delivery substance or agent, so as to increase the rate at which the delivery substance permeates through the skin. “Permeation enhancer”, “enhancer”, “penetration enhancer”, or similar terms refer to a material, or materials that achieve or facilitate such permeation enhancement, and an “effective amount” of an enhancer means an amount effective to enhance penetration through the skin, of huperzine, to a selected degree. An index of permeation enhancers is disclosed by David W. Osborne and Jill J. Henke, in their publication entitled Skin Penetration Enhancers Cited in the Technical Literature, published in “Pharmaceutical Technology” (June 1998), which may also be found at the worldwide web address known as: pharmtech.com/technical/osborne/osborne.htm, which is incorporated by reference herein. Enhanced permeation as affected through the use of such enhancers can be observed, for example, by measuring the rate of diffusion of the delivery substance through animal or human skin using a diffusion cell apparatus. Such a diffusion cell is described by Merritt et al., Diffusion Apparatus for Skin Penetration, J. of Controlled Released 61 (1984), incorporated herein by reference.

As used herein, “effective amount” refers to the minimal amount of a substance or agent, which is sufficient to achieve a desire effect. Therefore, when used in connection with huperzine, effective amount connotates an amount of huperzine sufficient to achieve a desired huperzine plasma level.

By the term “matrix”, “matrix system”, or “matrix patch” is meant a pre-determined amount of huperzine dissolved or suspended in a polymeric carrier or phase, in one aspect a pressure-sensitive adhesive, that can also contain other ingredients, or in which a permeation enhancer and other positive health benefit promoting substances may also dissolved or suspended. This definition is meant to include embodiments wherein such polymeric phase is laminated to a pressure sensitive adhesive or used within an overlay adhesive to form an adhesive matrix patch with a reservoir. A matrix system usually and preferably comprises an adhesive layer having an impermeable film backing laminated onto the distal surface thereof and, before transdermal application, a release liner on the proximal surface of the adhesive. The film backing protects the polymeric phase of the matrix patch and prevents release of the delivery substance and/or enhancer to the environment. The release liner function similarly to the impermeable backing, but is removed from the matrix patch prior to application of the patch to the skin as defined above. Matrix patches are known in the art of transdermal delivery to routinely contain such backing and release liner components, and matrix patches according to the present invention should be considered to comprise such backing and release liner or their functional equivalents. A matrix system therefore is a unit dosage form, or type of formulation, which includes a predetermined amount of huperzine, as well as other optional ingredients, such as good health-imparting ingredients, in a polymeric carrier, which optionally contains an enhancer. Examples without limitation, of adhesive matrix transdermal patches are those described or referred to in U.S. Pat. Nos. 5,122,383 and 5,460,820, which are incorporated by reference in their entirety.

As used herein, “liquid reservoir system,” its acronym “LRS,” or “liquid reservoir patch” refers to a transdermal delivery patch or system, in which huperzine and other optional ingredients, such as a permeation enhancer, are admixed with a carrier vehicle. The carrier vehicle comprises a fluid of desired viscosity, such as a gel or ointment, which is formulated for confinement in a reservoir having an impermeable backing and a skin contacting permeable membrane, or membrane adhesive laminate providing diffusional contact between the reservoir contents and the skin. For application, a peelable release liner is removed and the patch is attached to the skin surface. LRS patches are known in the art of transdermal drug delivery. Examples without limitation, of LRS transdermal patches are those described or referred to in U.S. Pat. Nos. 4,849,224, 4,983,395, which are incorporated by reference in their entirety.

As used herein, “inert carrier” refers to a polymeric carrier, or other carrier vehicle into which huperzine may be admixed in order to form a transdermal delivery formulation. Inert carriers must generally be pharmaceutically acceptable, in that they are suitable for administration to the skin without causing significant instances of adverse results. Further, inert carriers must not react with the active substance to substantially degrade it, or otherwise form impurities, which may be delivered to the skin.

As used herein, “hybrid,” “hybrid compound,” or “hybrid agent” refers to a new compound, which is synthesized by the addition of huperzine and another acetyl cholinesterase inhibitor. Examples of such hybrids include, but are not limited to huperzine-tacrine, huperzine-donepezil, huperzine-galantamine, etc.

As used herein, “topical formulation” refers to a chemical formulation in which huperzine may be incorporated, which is capable of being applied directly to the skin, and which does not include supporting structures such as backing films, etc. Examples of topical formulations without limitation include, gels, aerosols, creams, lotions, pastes, ointments, etc.

Concentrations, amounts, solubilities, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

For example, a concentration range of 0.1 to 30 ng/ml should be interpreted to include not only the explicitly recited concentration limits of 0.1 ng/ml and 30 ng/ml, but also to include individual concentrations within that range, such as 0.5 ng/ml, 0.7 ng/ml, 1.0 ng/ml, 5.2 ng/ml, 8.4 ng/ml, 11.6 ng/ml, 14.2 ng/ml, and sub-ranges such as 0.5-2.5 ng/ml, 4.8-7.2 ng/ml, 6-14.9 ng/ml, etc. This interpretation should apply regardless of the breadth of the range or the characteristic being described.

B. The Invention

The present invention encompasses a transdermally administered huperzine formulation for improving memory and cognitive function. In one aspect, the huperzine is administered in an amount sufficient to affect and maintain a blood plasma level of about 0.1 ng/mL to about 30 ng/mL. In another aspect, the blood plasma level may be about 1 ng/mL to about 15 ng/mL.

The time frame for achieving such blood plasma levels may be the result of such parameters as the type and size of the huperzine formulation, the amount of huperzine present in the formulation, and the flux rate achieved by the formulation. Further, the flux rate may be determined in part by the presence of specific types of penetration enhancers.

Elements such as patch size, huperzine content, enhancer amount, and enhancer type may all be coordinated in order to achieve the desired blood plasma levels within a desired amount of time. Others physiological factors, such as variations in individual skin type and permeability may effect the ultimate huperzine blood plasma level and the time frame in which it is achieved.

In one aspect, permeation rates of huperzine through living human skin may be in the range of about 0.01 ug/cm²/hr to about 15 ug/cm²/hr. In another aspect, therapeutic blood levels may be achieved in about 0.5-10 hours after initial formulation application. However, these general parameters are not limitations on the way in which the desired blood serum levels may be achieved. Different permeations, times, and amounts may be used to effect the desired blood levels by employing a formulation which produces different parameters.

By adjusting parameters such as the size and type of the transdermal formulation, the speed and duration of huperzine delivery may be varied. In one aspect of the present invention, a single dosage of the transdermal delivery formulation may achieve and sustain the huperzine plasma level of from about 0.1 to about 30 ng/ml for a duration of at least 3 days. In another aspect, a single dosage of the transdermal delivery formulation may achieve and sustain the huperzine plasma level of from about 0.1 to about 30 ng/ml for a duration of at least 7 days. In yet another aspect, the duration may be from about 24 hours to about 168 hours.

Specific huperzine delivery formulation types include but are not limited to: 1) topical formulations such as ointments, lotions, gels, pastes, mousses, aerosols, and skin creams; 2) transdermal patches such as adhesive matrix patches and liquid reservoir systems; 3) transmucosal tablets such as buccal or sublingual tablets or lozenges; and 4) suppositories. In short, any transdermal administration form is acceptable.

In one aspect, the huperzine delivery formulation may also include a permeation enhancer, or mixture of permeation enhancers in order to increase the permeability of the skin to huperzine. A wide range of known permeation enhancers have been found to enhance the delivery of huperzine and include but are not limited to: fatty acids, fatty acid esters, fatty alcohols, fatty acid esters of lactic acid or glycolic acid and their salts, amides, amines, pyrrolidones, glycerol triesters, terpenes, classical surfactants, organic acids, complexing agents, biologics, and mixtures thereof.

One enhancer that has been found to be unacceptable is Azone. Although Azone may provide penetration enhancement of various substances, the side effects experienced are considered intolerable. Particularly, Azone has been deemed unusable because of the severe skin irritation that results. Not only does Azone cause irritation to all layers of the epidermis, but also irritates all the dermis layers as well. Further, the skin irritation caused by Azone is irreversible damage, which results in alteration of the tissue and scarring.

Specific examples of acceptable fatty acids include but are not limited to, oleic acid, alkanoic acids, capric acid, hexanoic acid, lactic acid, lauric acid, linoleic acid and mixtures thereof.

Specific examples of acceptable fatty acid esters include but are not limited to methyl laurate, glycerol monooleate (GMO), sorbitan monooleate (SMO), glycerol monolaurate (GML), glycerol monolinoleate (GMLO), isopropyl myristate, isopropyl palmitate, methyl propionate, monoglycerides, propylene glycol monolaurate, sorbitan monolaurate, and mixtures thereof.

Specific examples of acceptable fatty alcohols include but are not limited to lauryl alcohol, caprylic alcohol, myristyl alcohol, cetyl alcohol, aliphatic alcohols, linolenyl alcohol, nerolidol, oleyl alcohol, and mixtures thereof.

Specific examples of acceptable fatty acid esters of lactic acid or glycolic acid or their salts include but are not limited to lauroyl glycolate, sodium lauryol glycolate, caproyl glycolate, sodium caproyl glycolate, cocyl glycolate, sodium cocyl glycolate, isostearoyl glycolate, tromethamine lauroyl glycolate, lauroyl lactylate, sodium lauroyl lactylate, caproyl lactylate, sodium caproyl lactylate, cocoyl lactylate, sodium cocyl lactylate, isostearoyl lactylate, tromethamine lauryol lactylate, and mixtures thereof.

Specific examples of acceptable amides include but are not limited to lauramide diethanolamide, alkanolamides, ethoxylated alkanolamides, ethylene bisamides, urea, and mixtures thereof.

Specific examples of acceptable pyrrolidones include but are not limited to N-methyl-pyrrolidone N-alkyl-pyrrolidones, pyrrolidone carboxylic acids, pyrrolidone carboxylic esters, and mixtures thereof.

Specific examples of acceptable glycerol triesters include but are not limited to triacetin, diacetin, monoacetin, tributylrin, tricaproin, tricaprylin, trilaurin, trymyristin, tripalmitin, tristearin, triethyl citrate, tributyl citrate, and mixtures thereof.

Specific examples of acceptable terpenes include but are not limited to lemonene, methone, pipertone, 1-8 cineole, terpineol, terpinen-4-ol pulegone, carvone, carveol, and mixtures thereof.

Specific examples of acceptable amines include but are not limited to lauryl-amine (dodecylamine), unsaturated cyclic ureas, urea, and mixtures thereof.

Specific examples of acceptable classical surfactants include, but are not limited to Brij surfactants, (such as Brij 30, Brij 36T, Brij, 35, Brij 52), Pluronic surfactants, (such as Pluronic F68, and Pluronic L62), Span surfactants, (such as Span 20 and Span 85), Tween surfactants, (Such as Tween 20, Tween 40, and Tween 80), Poloxomer surfactants, Myrj surfactants, bile salts, sodium laurate, sodium lauryl sulfate, and mixtures thereof.

Specific examples of acceptable complexing agents include but are not limited to cyclodextrine complexes and derivatives thereof, liposomes, microcapsules, microspheres, and mixtures thereof.

Specific examples of organic acids include, but are not limited to salicylic acid, citric acid, salicylates, and mixtures thereof.

Specific examples of acceptable biologics include but are not limited to L-α-amino acids, lecithin, phospholipids, and mixtures thereof.

In addition to those enhancer substances enumerated above, many natural substances are capable of acting as permeation enhancers. These natural substances include, but are not limited to: arecoline, berbamine, berberine, camphol, capsaicin, capsaicine, capsic acid, eucalyptus (oil), eucalyptols, ferulic acid, menthol, oleummenthae, paeonol, peppermint oil, tanshinone, and mixtures thereof.

In addition to huperzine, the formulation of the present invention may include additional cholinesterase inhibitors. In one aspect of the present invention, huperzine may be compounded with one or more specific other cholinesterase inhibitors to synthetically form a huperzine hybrid compound. Such hybrid compounds have been found to provide an increased, or synergistic cognitive function and memory enhancing effect, while minimizing the known side effects of many cholinesterase agents.

Specific examples of acceptable cholinesterase inhibitors which may be compounded to form a huperzine hybrid compound or agent, or which may be simply added to the transdermal formulation of the present invention include, but are not limited to Acricept (Donepezil), Galantamine, Metrifonate, Propentofylline, Rivastigmine (Exelon), Tacrine, Xanomeline, Astaxanthin, Celecoxib, Memantine, Selegiline, and mixtures thereof. In one aspect, the huperzine hybrid compound may be huperzine-tacrine.

The huperzine used in the formulation of the present invention may be any of the particular huperzine species recited in the definitions section, or a combination of two or more of such species. Further, huperzine may be combined with other positive health benefit conferring substances, or treatment agents, either before, during, or after its inclusion in the transdermal delivery formulation. Such positive health benefit conferring substances include but are not limited to vitamins, amino acids, minerals, herbal and botanical extracts, anti-oxidants, other materials which are essential to the body, and mixtures thereof.

Specific examples of acceptable vitamins include both water-soluble and oil soluble vitamins. Water-soluble vitamins include but are not limited to the B1, B2, B3, B4, B5, B6, B12, B13, B15, B17, biotin, choline, folic acid, inositol, para-amino benzoic acid (PABA), Vitamin C, Vitamin P, and mixtures thereof. Additionally, oil soluble vitamins include Vitamin A, Vitamin D, Vitamin E, Vitamin K and mixtures thereof.

Specific examples of acceptable amino acids include but are not limited to alanine arginine, carnitine, gamma-aminobutyric acid (GABA), glutamine, glycine, histidine, lysine, methionine, N-acetyl systeine, ornithine, phenylalanine, taurine, tyrosine, valine, and mixtures thereof.

Specific examples of acceptable minerals include but are not limited to calcium, potassium, iron, chromium, phosphorous, magnesium, zinc, copper and mixtures thereof, as well as any other minerals essential to the human body.

Specific examples of acceptable herbs and botanical extracts include but are not limited to Green tea plant, Causena Lansium, Crocus Sativus, Danshen (saliva miltiorrhize), Dongui (Radix angelicae sinesis), Eucommia, Evening primrose, Gastrodia elata, German chamomile, Ginseng, Gingko Baloba, Hopes, Horn goat weed (epimedium sagittatum), Kava, Lemon balm, Mishmi bitter (coptis sinesis), Morning star (Uncaria rhychophylla), Passion flower, Physostigmine, Securinega Suffructicosa, Scutellaria baicalensis, Siberian cork tree (phellodendron amurense), Skullcap, St. John's Wort, Valerian, and mixtures thereof.

Specific examples of acceptable antioxidants include but are not limited to polyphenols such as catechin, beta-carotene, coenzyme Q10, grapnel, and mixtures thereof.

In yet another aspect of the invention, the huperzine transdermal formulation may include one or more specific treatment agents, or drugs for treating other symptoms of particular disorders, which are related to the memory and cognitive function loss. In one aspect, the treatment agent may be an antipsychotic. In another aspect, the treatment agent may be an anxiolytic. In a further aspect, the treatment agent may be an antidepressant. In yet a further aspect, the treatment agent may be a hormone.

Specific examples of suitable antipsychotics include, but are not limited to: haloperidol, olanzapine, quietiapine, risperidone, and mixtures thereof.

Specific examples of suitable anxiolytics included, but are not limited to: alpraxolam, buspirone, diazepam, lorazepam, and mixtures thereof.

Specific examples of antidepressants include, but are not limited to: amitriptyline, bupropion, desipramimine, fluoxetine, fluvoxamine, nefazodone, nortriptyline, paroxetine, sertraline, trazodone, and mixtures thereof.

Specific examples of hormones include, but are not limited to: androgens, estrogens, dehydroepiandrostene (DHEA), melatonin, seratonin, and phosphatidyl serine.

The huperzine, other positive health benefit conveying substances, and other treatment agents may be either produced synthetically, or harvested from plants and other natural sources by methods such as extraction and concentration. In short, the source of the delivery substance may be either artificial, natural, or a combination thereof.

In one aspect, the transdermal delivery formulation of the present invention may be a topical formulation. As recited above, topical formulations may take a variety of specific forms, such as gels, ointments, pastes, aerosols, creams, lotions, and other hydrophobic or water-miscible vehicles. Other specific types of topical formulations not specifically mentioned will be readily recognized by those skilled in the art and fall within the purview of the present invention.

Specific examples of suitable hydrophobic and water-miscible agents include but are not limited, hydrocarbons (e.g. liquid paraffin, mineral oil, paraffin oil, white petrolatum, squalane), silicones (e.g. liquid polymethylsilaxanes, dimethicone), alcohols (e.g. ethanol, isopropyl alcohol, lauryl alcohol), polyols and polyglycols (e.g. propyl glycol, glycerin, triacetin, polyethylene glycols), Sterols (e.g. lanolin, cholesterol), carboxylic acids (e.g. lauric acid, oleic acid), esters and polyesters (e.g. ethylene glycol monostearate, sorbitan monoesters, glyceryl tristearate, olive oil, soybean oil, isopropyl myristate, isopropyl palmitate).

Specific examples of suitable emulsifiers include, but are not limited to sterols and sterol eaters (e.g. cholesterol), carboxylic acid salts (sodium, ethanol amine, etc. of lauric acid, oleic acid, etc.), esters and polyesters (e.g. ethylene glycol monoesters, propylene glycol monoesters, glycerol monoesters, sorbitan monoesters, sorbitol monoesters, polyoxyethylene esters, sorbitan diesters, polyoxy ethylene sorbitan polyesters—tweens), ethers and polyethers (e.g. polyethylene glycol monocetyl ethers, polyethylene-polypropylene glycols—pluronics), others (e.g. sodium lauryl sulfate, borax, ethanolamine).

Specific examples of suitable thickeners include, but are not limited to acrylate copolymers, algin, behenyl alcohol, 18-36 acid triglycerides, calcium carboxymethyl celluse, PVP/MA copolymers, carbomer (910, 934, 934p, 940, 941, 1342), carboxymethylcelluse sodium, cellulose, cetyl alcohol, guar gum, hydroxyethylcellulose, hydroxypropylcellulose, hydroxypropylmethylcellulose, methylcellulose, methyl hydroxyethylcellulose, PEGs, poloxamine (304, 504, 701, 904, 1102, 1304, 1502, etc.), polycarbophil, polyethylene, propylene glycol alginate, PVP, PVP/VA copolymer, silica, silicones, beeswax.

The transdermal delivery formulation of the present invention may take the form of an occlusive device, such as a transdermal patch, in order to provide a huperzine formulation. Such a transdermal patch may either be an adhesive matrix patch, a liquid reservoir system type patch, a buccal or sublingual tablet, lozenge, or the like.

In the case of the adhesive matrix patch, an amount of huperzine sufficient to produce the desired therapeutic blood plasma level is dissolved or suspended in a polymeric phase or carrier. A selected permeation enhancer, or mixture of enhancers may be included in the polymeric phase, as well as additional positive health benefit imparting substances as mentioned above. The size of an adhesive matrix patch may be adjusted to provide varying dosage amounts, and may vary from about 1 to 200 cm².

A wide range of adhesives useful in connection with transdermal patches will be known to those skilled in the art of transdermal drug delivery. In one aspect of the invention, acceptable adhesives may include polyacrylate polymers, rubber-based adhesives, and polysiloxanes adhesives.

In one aspect, polyacrylate polymers can be any of the homopolymers, copolymers, terpolymers, and the like of various acrylic acids. In another aspect of the invention, the acrylate polymers may be a combination of one or more monomers of acrylic acids and other copolymerizable monomers.

Acrylate polymers may also include copolymers of alkyl acrylates and/or methacrylates, and/or copolymerizable secondary monomers or monomers with functional groups. Specific examples of acrylate monomers, which are suitable for use with the present invention include, but are not limited to methacrylic acid, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecylmethacrylate, tridecyl acrylate, tridecyl methacrylate, and mixtures thereof.

Specific examples of functional monomers which are copolymerizable with the above-recited alkyl acrylates or methacrylates, which can also be used include, but are not limited to acrylic acid, methacrylic acid, maleic acid, maleic anhydride, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxethyl acrylate, methoxyethyl methacrylate, and mixtures thereof.

Further details and examples of acrylic adhesives which are suitable for use in the present invention are set forth in Satas, “The Handbook of Pressure-sensitive Adhesive Technology,” 2^{nd} ed,. Pp. 396-456 (1989), which is incorporated herein by reference in its entirety.

Examples of suitable acrylic adhesives which are commercially available include polyacrylate adhesives sold under the trademarks DUROTAK® by National Starch and Chemical Corporation, Bridgewater, N.J., as well as GELVA-MULTIPOLYMER SOLUTION Monsanto, St. Louis, Mo. Other examples of adhesives, and adhesive formulations, which can be used in connection with the present invention are disclosed in U.S. Pat. No. 5,656,286, which is incorporated herein by reference in its entirety.

In one aspect, utilizing a mixture of two or more acrylic polymers may facilitate sustained release of huperzine. Many variations and combinations of acrylics may be employed to achieve the desired increase in release duration. Examples of such combinations may be found in U.S. Pat. No. 6,024,976, which is incorporated herein by reference in its entirety. Other examples of such acrylic combinations will be readily recognized by those skilled in the art.

Specific examples of suitable rubber-based pressure sensitive adhesives include, but are not limited to hydrocarbon polymers, such as natural and synthetic polyisoprenes, polybutylenes and polyisobutylene (PIB), styrene/butadiene polymers, styrene-isoprene-styrene block copolymers, hydrocarbon polymers such as butyl rubber, halogen-containing polymers such as polyacrylic nitrile, polytetrafluoroethylene, polyvinyl chloride, polyvinylidene chloride, and polychlorodiene, and polysiloxanes, and other copolymers thereof.

Specific examples of suitable polysiloxanes include but are not limited to silicone pressure sensitive adhesives, which are a based on two major components: a polymer, or gum, and a tackifying resin. The polysiloxane adhesive may be prepared by cross-linking the gum, typically a high molecular weight polydiorganosiloxane with the resin to produce a three-dimensional silicate structure via a condensation reaction in an appropriate organic solvent. Various aspects of formulating polysiloxane adhesives are disclosed by Sobieski et al, in “Silicone Pressure sensitive Adhesives,” I.d. at Pp. 508-517.

Suitable silicone pressure-sensitive adhesives are commercially available and include the silicone adhesives sold under the trademarks BIO-PSA® Dow Corning Corporation, Medical Products, Midland, Mich.

In use, the matrix patch contains a distal backing and a proximal release liner laminated on the polymer layer. The distal backing defines the side of the matrix patch that faces the environment, (i.e., distal to the skin or mucosa), and the release liner is adhered to the proximal side and must be removed before patch application. The backing layer functions to protect the matrix polymer layer with the delivery substances and optional enhancer, and to provide an impenetrable layer that prevents loss of delivery substance to the environment. Thus, the material chosen for the backing should be compatible with the polymer layer, delivery substances, and enhancer, and should be minimally permeable to any components of the matrix patch.

Advantageously, the backing can be opaque to protect components of the matrix patch from degradation caused by exposure to ultraviolet light. Further, the backing should be capable of binding to and supporting the polymer layer, yet should be pliable to accommodate the movements of a person using the matrix patch.

Suitable materials for the backing include, but are not limited to: metal foils, metalized polyfoils, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene, and styrene-isoprene copolymers, polyethylene, and polypropylene. A thickness of about 0.0005 to about 0.01 inch is preferred. The release liner can be made of the same materials as the backing, or other suitable films coated with an appropriate release surface.

The matrix patch can further comprise various additives in addition to the polymer layer, delivery substances, and permeation enhancer that are the fundamental components of the adhesive matrix patch formulation. These additives are generally those pharmaceutically acceptable ingredients that are known in the art of transdermal substance delivery and, more particularly, in the art of transdermal substance delivery. However, such additive ingredients must not materially alter the basic and novel characteristics of the matrix patch. For example, suitable diluents can include mineral oil, low molecular weight polymers, plasticizers, and the like. Many transdermal delivery substance formulations have a tendency to irritate the skin after prolonged exposure thereto, thus addition of a skin irritation reducing agent aids may be desirable.

The LRS patch generally contains a backing layer having a reservoir portion configured to contain the carrier vehicle in which the huperzine is admixed or dissolved. Such carrier vehicles may be the same as those used for topical applications described above. Further, a micro porous membrane may be heat sealed across the opening of the reservoir in order to control the rate at which the huperzine is transmitted to the skin. Additionally, an adhesive layer will generally be applied to a portion of the backing layer surrounding the reservoir for adhering the LRS patch to the skin. Further, a release liner that is removed prior to application is placed upon the adhesive to prevent adhesion of the patch prior to application.

In use, the release liner is removed, and the patch is adhered to the skin at a selected application situs. When the contents of the reservoir have been depleted, the patch may be removed.

### C. EXAMPLES AND EXPERIMENTALS

The following examples of adhesive matrix formulations having a variety of huperzine containing compositions are provided to promote a more clear understanding of the possible combinations of the present invention, and are in no way meant as a limitation thereon.

In vitro human cadaver skin flux studies were conducted using modified Franz non-jacketed permeation cells. The temperature of the skin surface was maintained at 32° C. by placing the cells in a circulating water bath positioned over a stirring module. The epidermal membrane was separated from the human cadaver whole skin by the heat-separation method of Kligman and Christopher (Arch. Dermatol. 88:702 (1963)) involving the exposure of the full thickness skin to 60° C. heat for 60 seconds, after which time the stratum corneum and the epidermis (epidermal membrane) were gently peeled off the dermis.

For matrix skin flux study, the heat separated human epidermal membrane was cut into rectangular strips. The matrix was cut into 0.71 cm² circular discs. The release liner was peeled and discarded and the matrix disc was laminated onto the stratum corneum surface of the epidermal membrane. The skin-matrix sandwich was then loaded onto the diffusion cells. Each piece of the skin matrix sandwich was loaded between the donor and receiver compartments of a diffusion cell, with the epidermal side facing the receiver compartment, and clamped in place. The receiver compartment was then filled with 0.02% sodium azide aqueous solution. The solubility of the drug in this medium is adequate to ensure sink conditions throughout the experiment. The diffusion cell was then placed in a circulating water bath calibrated to maintain the skin surface temperature at 32±1° C. At predetermined sampling intervals, the entire contents of the receiver compartment were collected for drug quantitation and the receiver compartment was filled with fresh receiver solution, taking care to eliminate any air bubbles at the skin/solution interface.

For gel skin flux study, the epidermal membrane was cut and placed between two halves of the permeation cell with the stratum corneum facing the donor compartment. The skin was allowed to hydrate at 32° C. overnight with 0.02% (w/v) sodium azide solution in the receiver compartment. The following morning, 75 μl of a gelled formulation was placed into a cavity created by placing a Teflon washer over the stratum corneum surface. The cavity was then occluded by clamping an occlusive backing over the Teflon washer and gel. A 0.02% sodium azide aqueous solution was placed in the receiver compartment in contact with the dermal side of the epidermis, to ensure sink conditions for the drug. At predetermined sampling intervals, the entire contents of the receiver compartment were collected for drug quantitation and the receiver compartment was filled with fresh receiver solution, taking care to eliminate any air bubbles at the skin/solution interface.

The cumulative amount of drug permeated per unit area at any time t (Qₜ, ug/cm²) was determined as follows:

$${Q}_{t} = {\sum }_{n = 0}^{t} ( {C}_{n} * V ) / A$$

where Cₙ is the concentration (μg/ml) of the drug in the receiver sample for the corresponding sample time, V is the volume of fluid in the receiver chamber (˜6.3 cm³), and A is the diffusion area of the cell (0.64 cm²). The slope of the best fit line to the Qₜ vs. t plot gives the steady state flux (Jₛₛ, μg/cm²/hr); the intercept of this line on the time axis give the lag time (t_{L}, h).

Examples I-III include skin flux results from various embodiments of a transdermal matrix system according to the present invention containing Huperzine A.

### Example I

| | Composition | Qₜ (t = 24) |
|---|---|---|
| Formulation | (%, w/w) | (μg/cm²/t)* |
| Adhesive/HupA | 95/5/0 | 62.06 ± 19.66 |
| Adhesive/HupA/Triacetin | 85/5/10 | 92.55 ± 37.08 |
| Adhesive/HupA/SMO | 85/5/10 | 73.58 ± 19.17 |

Adhesive: pressure sensitive acrylic copolymers;
HupA: Huperzine A;
SMO: Sorbitan Monooleate.
*(Mean ± SD), n = 3 skins, 12 cells.

### Example II

| | Composition | Qₜ (t = 24) |
|---|---|---|
| Formulation | (%, w/w) | (μg/cm²/t)* |
| Adhesive/HupA | 97.5/2.5/0 | 77.06 ± 26.33 |
| Adhesive/HupA/L-DEA | 87.5/2.5/10 | 150.84 ± 35.33 |
| Adhesive/HupA/GMO/LA | 87.5/2.5/10 | 141.47 ± 33.04 |

Adhesive: pressure sensitive acrylic copolymers;
HupA: Huperzine A;
L-DEA: Lauromide DEA;
GMO: Glycerol monooleate;
LA: Lauryl alcohol.
*(Mean ± SD), n = 3 skins, 12 cells.

### Example III

| | Composition | Qₜ (t = 24) |
|---|---|---|
| Formulation | (%, w/w) | (μg/cm²/t)* |
| Adhesive/HupA | 97.5/2.5/0 | 67.81 ± 25.28 |
| Adhesive/HupA/Oleic acid | 87.5/2.5/10 | 90.86 ± 17.42 |
| Adhesive/HupA/Cineole | 87.5/2.5/10 | 91.42 ± 29.33 |

Adhesive: pressure sensitive acrylic copolymers.
*(Mean ± SD), n = 3 skins, 12 cells.

The above results show that using one or more penetration enhancer may significantly increase the skin flux of huperzine A when compared to a mixture of only huperzine A and an adhesive matrix as a control. A wide variety of acrylic polymers may be used to obtain similar results, as will be recognized by those skilled in the art.

### Example IV

Other formulations of transdermal matrix systems containing Huperzine may be formulated as follows.

| | Composition (%, w/w) |
|---|---|
| Formulation IV-1 | |
| Acrylic Adhesives | 50-99.5 |
| Huperzine | 0.01-20 |
| Enhancers | 0.01-20 |
| Formulation IV-2 | |
| PIB Adhesives | 50-99.5 |
| Huperzine | 0.01-20 |
| Enhancers | 0.01-20 |
| Formulation IV-3 | |
| Silicone Adhesives | 50-99.5 |
| Huperzine | 0.01-20 |
| Enhancers | 0.01-20 |
| Formulation IV-4 | |
| Acrylic Adhesive 1 | 1-99.5 |
| Acrylic Adhesive 2 | 1-99.5 |
| Huperzine | 0.01-20 |
| Enhancers | 0.01-20 |
| Formulation IV-5 | |
| Acrylic Adhesive | 1-99.5 |
| PIB Adhesive | 1-99.5 |
| Huperzine | 0.01-20 |
| Enhancers | 0.01-20 |
| Formulation IV-6 | |
| Acrylic Adhesive | 1-99.5 |
| Silicone Adhesive | 1-99.5 |
| Huperzine | 0.01-20 |
| Enhancers | 0.01-20 |
| Formulation IV-7 | |
| Silicone Adhesive | 1-99.5 |
| PIB Adhesive | 1-99.5 |
| Huperzine | 0.01-20 |
| Enhancers | 0.01-20 |
| Formulation IV-8 | |
| Eudragit Adhesive* | 50-99.5 |
| Huperzine | 0.01-20 |
| Enhancer | 0.01-20 |
| Plasticizers/Tackifiers | 0.01-20 |

*A single Eudragit or mixture of different grades of Eudragits (e.g. NE 30 D, L100, L12/5, S 100, S12/5, L 30 D-55, L100-55, E 100, E12/5, RL 100, RL 12/5, R100, RL PO, RL PM, RL 30 D, RS 100, RS 12/5, RS PM, RS PO, RS 30 D.)

### Example V

Gel formulations containing 10 mg/ml huperzine A, 3% Hydroxypropyl Methylcellulose and penetration enhancers were also evaluated according to the above-recited protocols.

| | Composition | Qₜ (t = 24) |
|---|---|---|
| Formulation | (%, v/v) | (μg/cm²/t)* |
| EtOH/H2O | 65/35 | 329.82 ± 230.46 |
| EtOH/H2O/GMO/LA | 65/30/2.5/2.5 | 1022.04 ± 226.38 |
| EtOH/H2O/L-DEA | 65/30/5 | 839.90 ± 352.62 |

EtOH = Ethanol, GMO: Glyceryl monooleate; LA: Lauryl alcohol; L-DEA: Lauromide DEA.
*(Mean ± SD), n = 3 skin3, 12 cells.

These examples show that penetration enhancers may enhance the flux of huperzine A from gel type formulations. Such gel formulations may either be used as a topical application or in a LRS patch.

### Example VI

In accordance with the present invention, a hybrid transdermal system may be employed for delivering huperzine. Such a hybrid system generally contains a polymeric, or other type of reservoir with an adhesive overlay. Bioactive agents may be contained in both the reservoir and the adhesive layer. A wide variety of substances may be used for the reservoir, and include, but are not limited to polymers (including adhesives), solutions, gels, emulsified gels, lotions and creams. Other variations of such a hybrid patch, as well as other particular substances for both the adhesive layer and reservoir will be readily recognized by those skilled in the art. Examples of such hybrid transdermal systems in accordance with the present invention may be as follows.

| | Composition |
|---|---|
| | (%, w/w) |
| Formulation VI-1 | |
| Matrix | |
| Acrylic Adhesives | 50-99.5 |
| Huperzine | 0-20 |
| Enhancers | 0-20 |
| Gel | |
| Ethanol | 0.1-99.5% |
| Propylene Glycol | 0-50% |
| Glycerin | 0-50% |
| Water | 0.1-99.5% |
| Enhancers | 0.01-20% |
| Huperzine | 0.01-20% |
| Gelling agents | 0-6% |
| Formulation VI-2 | |
| Matrix | |
| PIB Adhesives | 50-99.5 |
| Huperzine | 0.01-20 |
| Enhancers | 0.01-20 |
| Gel | |
| Ethanol | 0.1-99.5% |
| Propylene Glycol | 0-50% |
| Glycerin | 0-50% |
| Water | 0.1-99.5% |
| Enhancers | 0.01-20% |
| Huperzine | 0.01-20% |
| Gelling agents | 0-6% |
| Formulation VI-3 | |
| Matrix | |
| Silicone Adhesives | 50-99.5 |
| Huperzine | 0.01-20 |
| Enhancers | 0.01-20 |
| Gel | |
| Ethanol | 0.1-99.5% |
| Propylene Glycol | 0-50% |
| Glycerin | 0-50% |
| Water | 0.1-99.5% |
| Enhancers | 0.01-20% |
| Huperzine | 0.01-20% |
| Gelling agents | 0-6% |

### Example VII

Huperzines can be formulated with other positive health benefit-imparting substances. The following are a few examples of possible huperzine formulations containing such positive health benefit-imparting substances.

| | Composition (%, w/w) |
|---|---|
| Formulation VII-1 | |
| Acrylic Adhesive | 50-99.5 |
| Huperzine | 0.01-20 |
| Enhancers | 0.01-20 |
| Vitamin E* | 0.01-20 |

*One or more vitamins can be selected from either water-
soluble (e.g. Vitamin B1, B2, B3, B5, B6, B12, B13, B15, B17,
Biotin, Choline, Folic acid, Inositol, PABA, Vitamin C, and
Vitamin P) or oil soluble vitamins (e.g. Vitamins A, D, E and K).

| | |
|---|---|
| Formulation VII-2 | |
| Acrylic Adhesive | 50-99.5 |
| Huperzine | 0.01-20 |
| Enhancers | 0.01-20 |
| Amino Acids* | 0.01-20 |

*Amino acids are selected from but not limited to Alanine,
Arginine, Carnitine, DLPA, GABA, Glutamine, Glycine,
Histidine, Lysine, Methionine, N-Acetyl Cysteine, Ornithine,
Phenylalanine, Taurine, Tyrosine, and Valine.

| | |
|---|---|
| Formulation VII-3 | |
| Acrylic Adhesive | 50-99.5 |
| Huperzine | 0.01-20 |
| Enhancers | 0.01-20 |
| Minerals* | 0.01-20 |

*One or more minerals necessary to human body can be
selected.

| | |
|---|---|
| Formulation VII-4 | |
| Acrylic Adhesive | 50-99.5 |
| Huperzine | 0.01-20 |
| Enhancers | 0.01-20 |
| Herb/botanical extracts* | 0.01-30 |

*Herb/botanical extracts or isolated ingredients, which are
good for memory and aging, can be selected from but not
limited to Clausena lansium, Crocus sativus, Danshen (salvia
miltiorrhize), Dongui (Radix angelicae sinensis), Eucommia,
Evening primrose, Gastrodia elata, German chamomile, Ginseng,
Ginkgo Biloba, Hops, Horn goat weed (epimedium sagittatum),
Kava, Lemon balm, Mishmi bitter (coptis sinensis), Morning
star (uncaria rhynchophylla), Passion flower, Physostigmine,
Securinega suffruticosa, Scutellaria baicalensis, Siberian
cork tree (phellodendron amurense), Skullcap, St. John's Wort,
Valerian, etc.

| | |
|---|---|
| Formulation VII-5 | |
| Acrylic Adhesive | 50-99.5 |
| Huperzine | 0.01-20 |
| Enhancers | 0.01-20 |
| Anti-oxidant* | 0.01-20 |

*Anti-oxidant agents can be selected from but not limited to
Beta-carotene, Co-enzyme Q-10, Grapnol, etc.

| | |
|---|---|
| Formulation VII-6 | |
| Acrylic Adhesive | 50-99.5 |
| Huperzine | 0.01-20 |
| Enhancers | 0.01-20 |
| Melatomin | 0.01-20 |
| Formulation VII-7 | |
| Acrylic Adhesive | 50-99.5 |
| Huperzine | 0.01-20 |
| Enhancers | 0.01-20 |
| Phosphatidyl Serine | 0.01-20 |
| Formulation VII-8 | |
| Acrylic Adhesive | 50-99.5 |
| Huperzine | 0.01-20 |
| Enhancers | 0.01-20 |
| DHEA (Dehydroepiandrosterone) | 0.01-20 |
| Formulation VII-9 | |
| Acrylic Adhesive | 50-99.5 |
| Huperzine | 0.01-20 |
| Enhancers | 0.01-20 |
| Acetylcholinesterase inhibitors* | 0.01-20 |

*Acetylcholinesterase (AchE) inhibitors are selected but not
limited to Astaxanthin, Celecoxib, Donepezil, Galantamine,
Memantine, Metrifonate, Propentofylline, Rivastigmine,
Tacrine, Selegiline, Xanomeline, etc.

| | |
|---|---|
| Formulation VII-10 | |
| Acrylic Adhesive | 50-99.5 |
| Huperzine | 0.01-20 |
| Enhancers | 0.01-20 |
| Antiolytics* | 0.01-20 |

*Antiolytics can be selected from but not limited to
Alprazolam, Buspirone, Diazepam, and Lorazepam.

| | |
|---|---|
| Formulation VII-11 | |
| Acrylic Adhesive | 50-99.5 |
| Huperzine | 0.01-20 |
| Enhancers | 0.01-20 |
| Antidepressants* | 0.01-20 |

*Antidepressants can be selected from but not limited to
Amitriptyline, Bupropion, desipramine, Fluoxetine, Fluoxetine,
Nefazodone, Nortriptyline, Paroxetine, Sertraline, and
Trazodone.

| | |
|---|---|
| Formulation VII-12 | |
| Acrylic Adhesive | 50-99.5 |
| Huperzine | 0.01-20 |
| Enhancers | 0.01-20 |
| Antipsychotics* | 0.01-20 |

*Antipsychotics can be selected from but not limited to
Haloperidol, Olanzapine, Quetiapine, and Risperidone.

### Example VIII

The following examples illustrate a few of the possible topical preparations for huperzines in accordance with the present invention. Topical formulations, such as, gels, creams, lotions, ointments, paste, mousses, aerosols, etc., may be used to so long as when applied to the desired area of the skin the formulation will stay in place. Further, such formulations may be utilized in connection with an LRS patch.

1. Gel

| Formulation VIII-1 | Composition (%, w/w) |
|---|---|
| Huperzine | 0.01-20% |
| Ethanol | 0-70% |
| Propylene Glycol | 0-50% |
| Water | 0-95% |
| Glycerin | 0-50% |
| Enhancers | 0-20% |
| Gelling Agents/thickeners | 0.1-6% |

2. Cream (o/w)

| | Composition |
|---|---|
| Formulation VIII-2 | (%, w/w) |
| Huperzine | 0.01-20% |
| Stearyl Alcohol | 0.1-30% |
| Beeswax | 0.1-20% |
| Sorbitan Monooleate | 0.1-10% |
| Polysorbate 80 | 0.1-10% |
| Methyl Paraben | 0.01-2% |
| Propyl Paraben | 0.01-2% |
| Water | 40-95% |

3. Cream (w/o)

| | Composition |
|---|---|
| Formulation VIII-3 | (%, w/w) |
| Huperzine | 0.01-20% |
| Stearyl Alcohol | 1-30% |
| White Wax | 1-30% |
| Almond Oil | 10-80% |
| Sodium Borate | 0.1-5% |
| Water | 1-50% |

4. Vanishing Cream

| | Composition |
|---|---|
| Formulation VIII-4 | (%, w/w) |
| Huperzine | 0.01-20% |
| Stearic Acid | 0.1-30% |
| Stearyl Alcohol | 0.1-10% |
| Cetyl Alcohol | 0.1-10% |
| Glycerin | 1-30% |
| Methyl Paraben | 0.01-2% |
| Propyl Paraben | 0.01-2% |
| Potassium Hydroxide | 0.01-3% |
| Water | 40-95% |

5. Lotion

| | Composition |
|---|---|
| Formulation VIII-5 | (%, w/w) |
| Huperzine | 0.01-20% |
| White Petrolatum | 0.1-10% |
| Mineral Oil | 0.1-10% |
| Propylene Glycol Stearate | 0.1-10% |
| Stearyl Alcohol | 0.1-10% |
| Benzyl Alcohol | 0.01-5% |
| Propylene Glycol | 0.1-20% |
| Ethanol | 0.1-50% |
| Water | 40-95% |

6. Ointment

| | Composition |
|---|---|
| Formulation VIII-6 | (%, w/w) |
| Huperzine | 0.01-20%% |
| White Petrolatum | 50-95% |
| White Wax | 0.1-10% |
| Stearyl Alcohol | 0.1-10% |
| Cholesterol | 0.1-10% |

7. Water-washable Ointment

| | Composition |
|---|---|
| Formulation VIII-7 | (%, w/w) |
| Huperzine | 0.01-20% |
| White Petrolatum | 1-50% |
| Stearyl Alcohol | 1-50% |
| Propylene Glycol | 1-30% |
| Sodium Lauryl Sulfate | 0.01-5% |
| Methyl Paraben | 0.01-2% |
| Propyl Paraben | 0.01-2% |
| Water | 1-40% |

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

1-52. (canceled)

53. A transdermal formulation for improving memory and cognitive function comprising:
an inert carrier having from about 0.01% w/w to about 20% w/w of huperzine admixed therewith, and including a permeation enhancer selected from the group consisting of: fatty acids, fatty acid esters, fatty alcohols, amides, pyrrolidones, glycerol triesters, terpenes, their salts, and mixtures thereof, wherein said formulation provides a huperzine blood plasma level of from about 0.1 ng/ml to about 30 ng/ml, upon administration to a subject.

54. The transdermal formulation of claim 53, wherein the blood plasma level attained is from about 0.5 to about 15 ng/ml.

55. The transdermal formulation of claim 53, wherein the blood plasma level is achieved within about 0.5 to about 48 hours after administration of the formulation.

56. The transdermal formulation of claim 53, wherein a single dose is sufficient to sustain the huperzine blood plasma level for a duration of at least about 3 days.

57. The transdermal formulation of claim 53, wherein a single dosage is sufficient to sustain the huperzine blood plasma level for a duration at least about 7 days.

58. The transdermal formulation of claim 53, wherein the huperzine is a member selected from the group consisting of huperzine A, huperzine B, huperzine X, and salts, analogs, derivatives, prodrugs, and mixtures thereof.

59. The transdermal formulation of claim 58, wherein the huperzine is huperzine A.

60. The transdermal formulation of claim 58, wherein the huperzine is huperzine B.

61. The transdermal formulation of claim 58, wherein the huperzine is huperzine X.

62. The transdermal formulation of claim 53, wherein the inert carrier comprises a pressure sensitive adhesive, and the formulation is an adhesive matrix patch.

63. The transdermal formulation of claim 53, wherein the inert carrier is a liquid reservoir, and the formulation is a liquid reservoir system.

64. The transdermal formulation of claim 53, wherein the formulation is a topical formulation.

65. The transdermal formulation of claim 53, wherein the permeation enhancer is selected from the group consisting of: a terpene compound, lauromide DEA, glycerol monooleate, sorbitan monooleate, lauryl alcohol, triacetin, cineole, oleic acid, and mixtures thereof.

66. The transdermal formulation of claim 53, wherein said huperzine further comprises a huperzine hybrid compound.

67. The transdermal formulation of claim 66, wherein said huperzine hybrid compound is a huperzine-tacrine hybrid.

68. The transdermal formulation of claim 53, further comprising a hormone admixed with the carrier.

69. The transdermal formulation of claim 53, wherein the hormone is a member selected from the group consisting of estrogens, androgens, melatonin, seratonin, DHEA, phosphatidyl serine, and mixtures thereof.

70. The transdermal formulation of claim 69, wherein the hormone is estrogen.

71. The transdermal formulation of claim 53, further comprising a treatment agent selected from the group consisting of antipsychotics, anxiolytics, antidepressants, and mixtures thereof.

72. The transdermal formulation of claim 71, wherein the treatment agent is an antipsychotic.

73. The transdermal formulation of claim 71, wherein the treatment agent is an anxiolytic.

74. The transdermal formulation of claim 71, wherein the treatment agent is an antidepressant.

75. The transdermal formulation of claim 53, further including a positive health benefit imparting substance selected from the group consisting of: vitamins, amino acids, anti-oxidants, and mixtures thereof.

76. The transdermal formulation of claim 75, wherein the positive health benefit imparting substance is a vitamin.

78. The transdermal formulation of claim 75, wherein the positive health benefit imparting substance is an amino acid.

79. The transdermal formulation of claim 75, wherein the positive health benefit imparting substance is an anti-oxidant.

80. A transdermal formulation for improving memory and cognitive function consisting essentially of:
a mixture of an inert carrier and huperzine in an amount of from about 0.01% w/w to about 20% w/w, which provides a huperzine blood plasma level of from about 0.1 to about 30 ng/ml upon administration to a subject.

81. A method of improving memory and cognitive function in a subject, comprising:
transdermally administering a huperzine formulation to the subject which provides a huperzine blood plasma level of from about 0.1 to about 30 ng/ml.

82. The method of claim 81, wherein the huperzine is a member selected from the group consisting of huperzine A, huperzine B, huperzine X, and salts, analogs, derivatives, prodrugs, and mixtures thereof.

83. The method of claim 81, wherein the blood plasma level is from about 0.5 to about 15 ng/ml.

84. The method of claim 81, wherein the huperzine blood plasma level is attained within about 0.5 to about 48 hours after initiation of the huperzine administration.

85. The method of claim 81, wherein the huperzine blood plasma level is sustained for a duration of at least 3 days from a single transdermal administration.

86. The method of claim 81, wherein the huperzine blood plasma level is sustained for a duration of at least 7 days from a single transdermal administration.

87. The method of claim 81, further comprising a hormone.

88. The method of claim 81, wherein the hormone is a member selected from the group consisting of estrogens, androgens, melatonin, seratonin, DHEA, phosphatidyl serine, and mixtures thereof.

89. The method of claim 88, wherein the hormone is estrogen.

90. The method of claim 81, further comprising a treatment agent selected from the group consisting of antipsychotics, anxiolytics, antidepressants, and mixtures thereof.

91. The method of claim 90, wherein the treatment agent is an antipsychotic.

92. The method of claim 90, wherein the treatment agent is an anxiolytic.

93. The method of claim 90, wherein the treatment agent is an antidepressant.

94. The method of claim 90, further comprising co-administering a positive health benefit imparting substance selected from the group consisting of: vitamins, amino acids, anti-oxidants, and mixtures thereof.

95. The method of claim 94, wherein the positive health benefit imparting substance is a vitamin.

96. The method of claim 94, wherein the positive health benefit imparting substance is an amino acid.

97. The method of claim 94, wherein the positive health benefit imparting substance is an anti-oxidant.
